(19) United States
(12) Patent Application Publication
(10) Pub. No.: US 2005/0107709 A1
(43) Pub. Date: May 19, 2005

(54) Method and arrangement for optically measuring swelling of the nose

(75) Inventors: Uwe Hampel, Dresden (DE); Eckhard Schleicher, Dresden (DE); Gunter Eike Wustenberg, Dresden (DE); Uwe Oehmichen, Dresden (DE); Karl-Bernd Huttenbrink, Dresden (DE)

Correspondence Address: GREENBLUM & BERNSTEIN, P.L.C., 1950 ROLAND CLARKE PLACE, RESTON, VA, 20191, US

(73) Assignee: TECHNISCHE UNIVERSITAT DRESDEN, Dresden (DE)

(21) Appl. No.: 10/954,292

(22) Filed: Oct. 1, 2004

Related U.S. Application Data
(63) Continuation of application No. PCT/DE03/01146, filed on Apr. 1, 2003.

(30) Foreign Application Priority Data
Apr. 2, 2002 (DE) 102 15 212.8

(51) Int. Cl. A61B 6/00

(52) U.S. Cl. 600/476

(57) ABSTRACT

Arrangement and method for optically measuring swelling of a nose. The arrangement includes a device having at least one light-producing component, at least one light-detecting component, an emitter electronics system, a receiver electronics system, and a controller. At least one optical emitter device is connected to the device via at least one first optical connection. At least one optical receiver device is connected to the device via at least one second optical connection. The at least one optical emitter device and the at least one optical receiver device are arranged on an application device. This Abstract is not intended to define the invention disclosed in the specification, nor intended to limit the scope of the invention in any way.

### CROSS-REFERENCE TO RELATED APPLICATIONS

The instant application is a continuation of International Application No. PCT/DEO3/01146 filed on Apr. 1, 2003 and published as International Publication WO 03/082089 on Oct. 9, 2003, the disclosure of which is hereby expressly incorporated by reference hereto in its entirety. The instant application also claims priority under 35 U.S.C. §119 of German Application No. 102 15 212.8 filed on Apr. 2, 2002.

### BACKGROUND OF THE INVENTION

1. Field of the Invention

The invention relates to the fields of medicine and device construction and relates to a method and an arrangement for optically measuring swelling of the nose, which can be used to, e.g., measure nasal obstruction after allergen provocation.

2. Discussion of Background Information

From a medical point of view, there is a need for an objectification of the measurement of swelling, and of the course of swelling, e.g., in allergic reactions that are triggered by, e.g., the nasal provocation test.

Nowadays, the diagnosis of allergic rhinitis is made by calculating a symptom score (itchy nose, secretion, remote symptoms, such as watery eyes) and by measuring the nasal obstruction after allergen provocation with the aid of rhinomanometry (Clement et al.: Rhinomanometry—a review, ORL J. Otorhinolaryngol. Relat. Spec. 46, 173-91, 1984). The disadvantage of rhinomanometry is that the measurement cannot be carried out during the allergen application. With severe nasal obstruction, patients experience the rhinomanometry as a very unpleasant experience. Faulty measurements also occur frequently with uncooperative patients.

Another possibility for determining the swelling of the nose and, in particular, of the nasal mucous membrane is acoustic rhinometry (Fisher: Acoustic rhinometry, Clin. Otolaryngol. 22, 307-17, 1997). These measurements have a relatively large spread in their results. Moreover, sufficient precision is achieved only for the front sections of the nose. Additionally, no medication or allergen provocation can take place during the measurement. A continuous measurement is not possible with this method, either.

Furthermore, it is not possible with either method to say whether a nasal swelling is due to a change in the microcirculation or the formation of an edema.

### SUMMARY OF THE INVENTION

The invention relates a method and an arrangement for optically measuring swelling of the nose. As a result, a largely objective measurement of the swelling of the nose is rendered possible, in particular, while provocation tests are being conducted.

The arrangement according to the invention for optically measuring swelling of the nose includes a basic device with light-producing components and light-detecting components. The device also includes associated emitter and receiver electronic systems and controllers. Furthermore, at least one optical connection is utilized between the basic device and an optical emitter element, whereby the transmission of the light produced by the light-producing components provided by optical elements in the optical connection to the emitter element. Furthermore, at least one optical connection is present between an optical receiver element and the light-detecting components. Emitter and receiver elements, which are arranged on an application element, are located outside the basic device. The application element thus provides for an arrangement wherein the emitter and receiver elements makes it possible for the light emitted by the emitter element to pass through the swellable tissue of at least one side of the nose to the receiver element. Furthermore the application element can be placed in a form-lockiing manner at least on the upper part of the nose.

With the arrangement according to the invention, the swelling of the nasal tissue can be optically recorded. With the device, the nasal tissue is irradiated from outside by a light source that is emitted from an emitter element. The scattered light passing through the tissue is then recorded by a detector, which can have the form of a receiver element. The emitter and receiver can be arranged on either the same side of the nose or on the opposite sides of the nose. When passing through the nasal tissue, the light passes through a number of tissue layers, such as skin, musculature, mucous membrane, bone, cartilage, and the airways. A part of the tissue which is penetrated is characterized by swellability, in particular, the nasal mucous membrane located above the bone of the nasal concha. In the course of the swelling, an increase of the blood volume occurs in this part of the tissue due to the influx of blood into the cavernous bodies. The inflowing blood is primarily of an arterial nature and thus normally 95% saturated with oxygen. Furthermore, in the event of the formation of an edema, possibly associated with the swelling, an increase in the tissue fluid volume occurs. It is therefore advantageous to conduct the irradiation spectrometrically in order to be able to quantitatively record, separately, the volume proportions of the oxygenated and deoxygenated hemoglobin and of the tissue fluid. This can take place either by using a white-light source and a spectrometer detector (e.g., diode line spectrometer) or by using several light sources with discrete radiation spectra (e.g., LEDs and laser diodes).

Since the above-noted substances involved in the swelling have different optical absorption spectra, a separate absolute or relative determination of the volume proportions is possible with corresponding mathematical methods. Such an arrangement has hitherto not been described.

One advantage of the arrangement according to the invention is that it is characterized by a non-invasive application and/or measurement from the outside and requires simple handling.

The invention also provides for an arrangement for optically measuring swelling of a nose, wherein the arrangement comprises a device which includes at least one light-producing component, at least one light-detecting component, an emitter electronics system, a receiver electronics system, and a controller. At least one optical emitter device is connected to the device via at least one first optical connection. At least one optical receiver device is connected to the device via at least one second optical connection. The at least one optical emitter device and the at least one optical receiver device are arranged on an application device.

The at least one first optical connection may transmit light produced by the at least one light-producing component. The at least one light-producing component may produce light which is transmitted by the at least one first optical connection to the at least one optical emitter device. The at least one second optical connection may transmit light received by the at least one light-detecting component. The at least one light-detecting component may detect light which is transmitted by the at least one second optical connection to the at least one optical receiver device. The at least one light-producing component may comprise a plurality of light-producing components. The at least one optical emitter device and the at least one optical receiver device may be arranged external to the device. The application device may be structured and arranged to fit onto a nose of a person. The at least one optical emitter and receiver devices may be structured and arranged to allow the light emitted by the at least one optical emitter device to pass through swellable tissue of at least one side of the nose and to the at least one optical receiver device. The application device may be capable of being fixed in a form-locking manner to at least an upper part of a nose of a person.

The at least one light-producing component may comprise at least one light source. The at least one light source may comprise a plurality of light sources emitting light of different wavelengths. The at least one light-producing component may comprise at least one of an LED, a laser, and a halogen lamp. The at least one light-producing component may produce a constant optical output. The emitter electronics system may be structured and arranged to allow the at least one light-producing component to produce a constant optical output. The emitter electronics system may be structured and arranged to modulate an intensity of the at least one light-producing component. The emitter electronics system may be structured and arranged to provide for high-frequency modulation of the at least one light-producing component. The emitter electronics system may provide for light impulses which are less than or equal to a nanosecond.

The at least one light-detecting component may comprise at least one of at least one photodetector and at least one spectrometer detector. The at least one light-detecting component may comprise at least one of at least one photo semiconductor detector and at least one photomultiplier. The application device may comprise a clamp-shaped body. The clamp-shaped body may be rigid. The clamp-shaped body may be slightly flexible. The application device may comprises two separate elements. The application device may be capable of being adhesively attached to a nose of a person. The application device may be capable of being secured to a person via an expansible/length-adjustable strap running around a back of a head of the person. The application device may be capable of being secured to a spectacle frame.

The arrangement may further comprise a strap adjustably connected to the application device. The arrangement may further comprise at least one optical screening element capable of being arranged around a measuring field. The at least one optical screening element may comprise one of a light-impervious cap and an optical filter. The at least one optical emitter device may comprise at least two optical emitter elements and the at least one optical receiver device may comprise at least two optical receiver elements.

The at least one optical emitter device may comprise at least two optical emitter elements arranged on one side of the application device and the at least one optical receiver device may comprise at least two optical receiver elements arranged on another side of the application device.

The at least one optical emitter device comprises at least two optical emitter elements adapted to be arranged on one side of a nose of a person and wherein the at least one optical receiver device comprises at least two optical receiver elements adapted to be arranged on another side of the nose of the person.

The invention also provides for a method of optically measuring swelling of a nose using the arrangement described above, wherein the method comprises emitting light with the at least one light-producing component, transmitting the light, via the at least one first optical connection, from the at least one light-producing component to the at least one optical emitter device, allowing the light to penetrate through at least one side of the nose, capturing the light emerging from the nose with the at least one optical receiving device, and transmitting the light, via the at least one second optical connection, from the at least one optical receiver device to the at least one light-receiving component.

The method may further comprise recording incoming values that represent a time course of a spectral extinction of optical radiation produced by the at least one light-producing component at selected emission wavelengths before and during a natural swelling of swellable nasal tissue. The method may further comprise calculating diagnostically utilizable parameters from the incoming values.

The method may further comprise recording incoming values that represent a time course of a spectral extinction of optical radiation produced by the at least one light-producing component at selected emission wavelengths before and during a provoked swelling of swellable nasal tissue. The method may further comprise calculating diagnostically utilizable parameters from the incoming values.

The at least one light-producing component may comprise different light-producing components producing different emission wavelengths, and the method may comprise switching, with the emitter electronics system, the different light-producing components, whereby the different light-producing components are switched on brightly one after another.

The method may further comprise measuring, before optically measuring swelling of the nose, a dark value with non-activated light-producing components to correct for ambient influences.

The method may further comprise detecting, while optically measuring swelling of the nose, an intensity measurement of the at least one light-producing component.

The method may further comprise simultaneously detecting, while optically measuring swelling of the nose, an intensity measurement of the at least one light-producing component, whereby the detecting detect an optical output power. The light may comprise light emitted from a single white-light source, whereby the light is used to differentiate different tissue components

The at least one light-producing component may comprise a single white-light source. The at least one optical receiving device may comprise a spectrometer detector. The at least one light-producing component may comprise a plurality of light-producing components, and the method may further comprise producing, with the light-producing components, light with different carrier frequencies and modulating an intensity of the light emitted by the light-producing components. The method may further comprise separately detecting, via frequency demultiplexing, light signals. The modulating may be sinusoidal.

The at least one light-producing component may comprise a plurality of light-producing components, and the method may further comprise emitting, with the light-producing components, high-frequency modulated light and demodulating light signals in order to determine a phase shift and an amplitude damping.

The at least one light-producing component may comprise a plurality of light-producing components, and the method may further comprise emitting, with the light-producing components, very short light pulses of less than or equal to one nanosecond and determining a temporal photon arrival distribution of received light signals.

The at least one light-producing component may comprise a plurality of light-producing components, and the method may further comprise emitting, with the light-producing components, very short light pulses of less than or equal to one nanosecond and determining, via an optical receiver element, a temporal photon arrival distribution of received light signals, where by measuring occurs in a time-resolved manner.

The optically measuring of the swelling of the nose may comprise one of optically measuring a swelling of each nostril separately and optically measuring a swelling of each side of the nose separately.

The at least one light-producing component may comprise a plurality of light-producing components, and the method may further comprise emitting, with the light-producing components, light of different wavelengths and transmitting the light of different wavelengths from the light-producing components to at least one optical emitter device.

The at least one optical emitter device may comprise a plurality of optical emitter devices, and the method may further comprise arranging the optical emitter devices on each side of the nose and transmitting the light from the at least one light-producing component to the optical emitter devices.

The invention also provides for a system for optically measuring swelling of a nose, wherein the system comprises an arrangement including at least one light-producing component, at least one light-detecting component, an emitter electronics device, a receiver electronics device, and a controller. At least one optical emitter device can be arranged on a portion of the nose. At least one optical receiver device can be arranged on another portion of the nose. The at least one optical emitter device is structured and arranged to emit light into the nose, and the at least one optical receiver device is structured and arranged to capture the light emitted by the at least one optical emitter device.

The invention also provides for a method of optically measuring swelling of a nose using the system described above, wherein the method comprises emitting light with the at least one light-producing component, transmitting the light from the at least one light-producing component to the at least one optical emitter device, allowing the light to penetrate through at least one side of the nose, capturing the light emerging from the nose with the at least one optical receiving device, and transmitting the light from the at least one optical receiver device to the at least one light-receiving component.

The invention also provides for a system for optically measuring swelling of a nose, wherein the system comprises an arrangement including at least one light-producing component, at least one light-detecting component, an emitter electronics device, a receiver electronics device, and a controller. At least one optical emitter device can be arranged on a portion of the nose. At least one optical receiver device can be arranged on another portion of the nose. A first connection device is used for transmitting light from the at least one light-producing component to the at least one optical emitter device. A second connection device is used for transmitting light from the at least one optical receiving device to the at least one light-receiving component. The at least one optical emitter device is structured and arranged to emit light into the nose and The at least one optical receiver device is structured and arranged to capture the light emitted by the at least one optical emitter device.

The invention also provides for a method of optically measuring swelling of a nose using the system described above, wherein the method comprises emitting light with the at least one light-producing component, transmitting the light from the at least one light-producing component to the at least one optical emitter device, allowing the light to penetrate through at least one side of the nose, capturing the light emerging from the nose with the at least one optical receiving device, and transmitting the light from the at least one optical receiver device to the at least one light-receiving component.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1a shows a front view of an application element;

FIG. 1b shows a top view of the application element of FIG. 1a;

FIG. 2a shows another embodiment of the application element with an active emitter element and an active receiver element;

FIG. 2b shows another embodiment of the application element with a passive emitter element and a passive receiver element;

FIG. 3 shows an application element mounting on the head and nose of user;

FIG. 4 shows a basic device which is coupled to the application element;

FIG. 5a shows a diagrammatic representation of a cross section of the nose before swelling and illustrates the position of the optical elements and the irradiation channel;

FIG. 5b shows a diagrammatic representation of a cross section of the nose after swelling and illustrates the position of the optical elements and the irradiation channel; and

FIG. 6 shows a diagrammatic representation of the measured extinction values in the course of swelling.

### DETAILED DESCRIPTION OF THE PRESENT INVENTION

The arrangement according to the invention comprises an application element 1 and at least one basic device 12 which includes an emitter electronics system 15 and a receiver electronic system 16 which are necessary to carry out the measuring task. The application element 1 is in direct contact with the nasal tissue during the measurement.

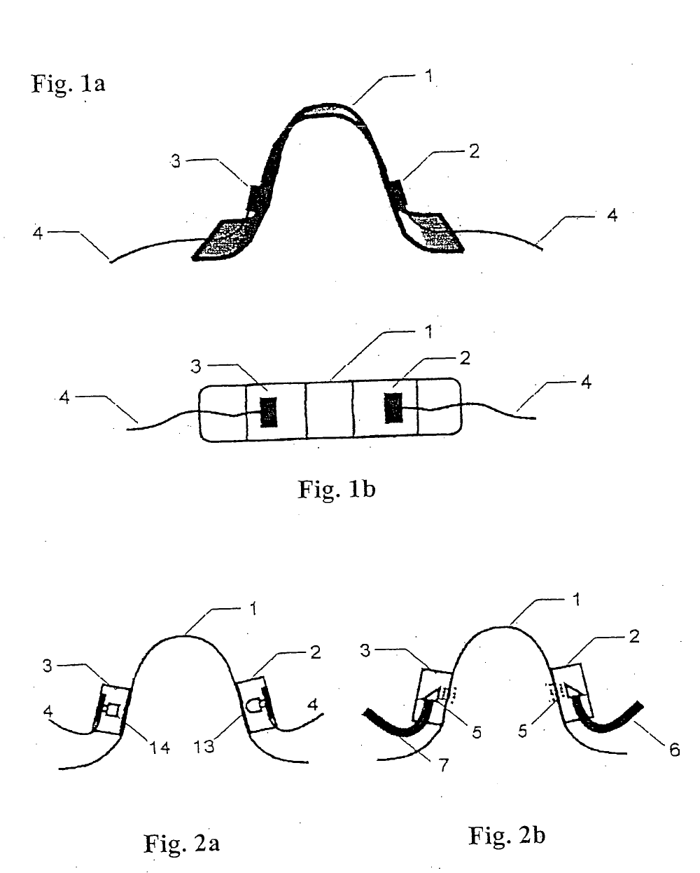

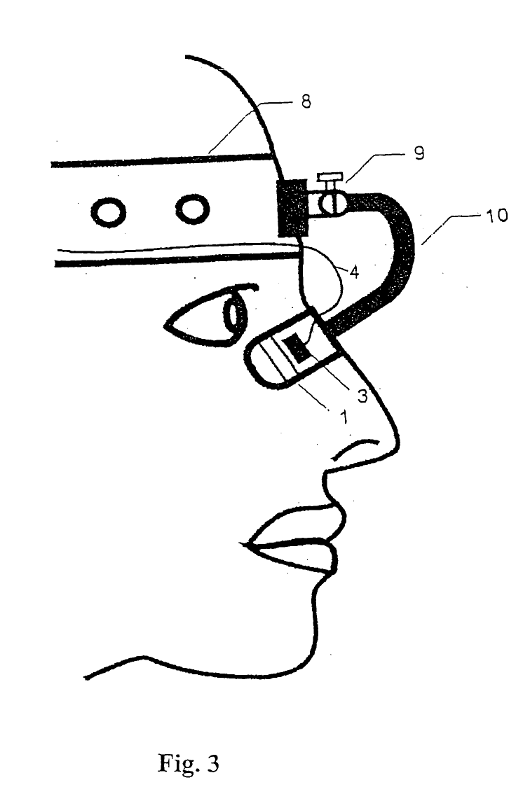

FIGS. 1a and 1b show one embodiment of the application element 1. The application element 1 has the form of a clamp-shaped base body. Both sides of the body can be placed on the sides of the nose in a form-locking manner (see FIG. 3). A light-emitting element 2, which can be an optical emitter element, is arranged on one side of the application element 1. A light-receiving element 3, which can be an optical receiver element, is arranged on the opposite side. As seen in FIG. 2a, these devices 2, 3 can be embodied as discrete radiation sources and detectors whose optical axes are aligned in the direction of the tissue, and which are connected to the basic device 12 via current-carrying cables 4. Alternatively, the devices 2, 3 can utilize optical connections 6, 7 which transfer light from and to the basic device 12. The devices 2, 3 can be oriented with their radiating surfaces perpendicular to the nasal tissue or can be aligned to the tissue by an arrangement of corresponding optical deflection elements (such as, e.g., mirrors and microprisms.

FIG. 3 illustrates how the application element 1 can be mounted on a user via a headband 8 which can be placed on the head. The headband 8 ensures a stable positioning of the application element I on the bridge of the nose during the measurement. The application element 1 is connected to the headband 8 via a clamp 10. This connection is embodied such that an exact positioning of the application element 1 on the bridge of the nose is possible via, e.g., a lockable ball joint 9 or a flexible metal hose. The invention also contemplates further relevant variants and/or arrangements of the invention as follows:
The application element 1 can be adhesively attached to the nose;
The application element 1 can be pressed directly onto the nose with the aid of an elastic belt. The belt may be adjustable in circumference to the size of the head;

The application element 1 can also be embodied as a spectacles-like frame that sits on the root of the nose so that the optical emitter and receiver elements are pressed onto the nasal tissue by gravity;
The arrangement according to the invention also contemplates that the emitter and receiver elements are arranged on two separate basic elements (pads) that are adhesively attached on each side of the nose separately from one another.

In order to provide for precise and reproducible measurement, in addition to the spatially stable and motion-free fixing of the application element 1 to the nose, it is important to suppress and/or calibrate out extraneous light influences. It is therefore advantageous to utilize optical filters in the arrangement. Alternatively, the measuring site should be covered during the measurement by a light-impervious cap, which can be, e.g., a plastic cap. The cap can also be fixed to the headband 8 as well, and can be closed over the field of measurement during the examination as required.

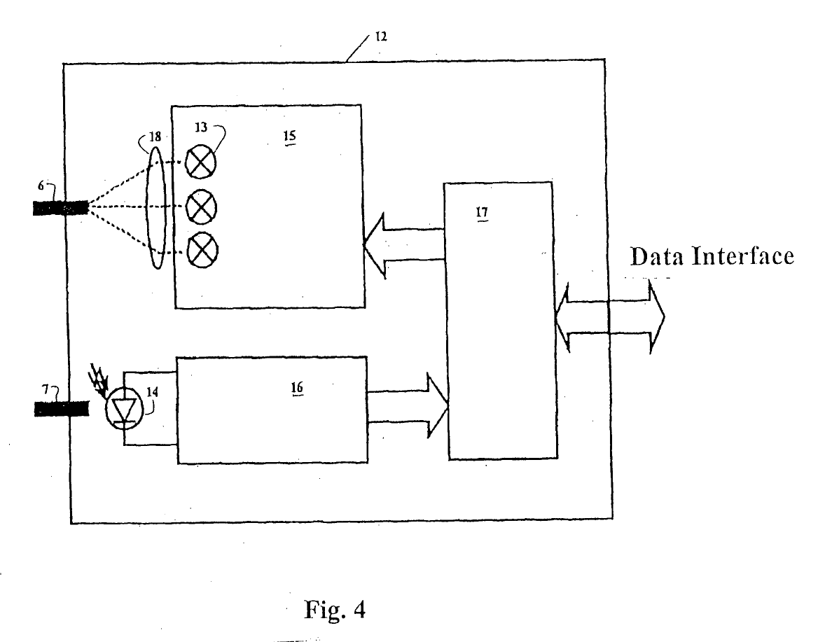

FIG. 4 shows a basic device 12 which includes a plurality of light-producing components 13 and one or more light-detecting components 14 arranged inside the device. The application element 1 described above can be connected to the device 12 via the optical connections 6, 7. The basic device 12 also includes an emitter electronics system 15 for the optical light-producing components 13, a receiver electronic system 16, and a controller 17 to which other devices can be connected via a data interface. The light-producing components 13 are arranged at the output of the emitter electronics system 15. An optical element 18 is used to concentrate the light from the light-producing components 13. In this way, the concentrated light is introduced into the optical connection 6.

The light-detection component 14 is connected to the input of the receiver electronics system 16. In this way, light enters from the optical connection 7 into the component 14.

A spectrometric measurement is advantageously used for the optical measurement of swelling and provides for the differentiation of the causes of swelling. Light sources with limited spectrum (LEDs, semiconductor lasers) and a photodetector that is adequately sensitive for the selected spectral range (semiconductor photodetector, photomultiplier) can be used for this measurement. Alternatively, a white-light source and a detector measuring in a spectrometrically resolving manner can be used. The object of the measurement is to detect light attenuation values (optical density of the tissue) at individual wavelengths of interest over time. This results from the equation:

$$E ( λ , t ) = {log}_{10} ( \frac{{I}_{S} ( λ , t )}{{λ}_{D} ( λ , t )} ) ,$$

where I_{S}(λ,t) denotes the light intensity radiated at the emitter element and I_{D}(λ,t) denotes the light intensity arriving at the receiver element at the wavelength λ and at the point of time t. In general the extinction E(λ,t) is a function of the light scattering and the light absorption in the tissue and thus provides a measured value for the geometric and optical change of the tissue. By performing the subtraction E(λ₁,t)−E(λ₂,t) at two wavelengths, a relative measurement of the change can be determined, which reflects the ratio of the volume change values of individual tissue constituents and which is largely free of geometric effects. Thus when using, e.g., a hemoglobin-sensitive wavelength of λ₁=800 nm and an H₂O-sensitive wavelength of λ2=970 nm, the ratio between the blood and tissue fluid increase can be shown. Furthermore, by using special optical measurement techniques, it is possible to separately determine the scatter and absorption properties of the tissue. To this end, photon time delay measurements are necessary with the aid of a high-frequency modulation technology (intensity modulation of the light source(s) and amplitude and phase measurement of the receiver signal) or a pulse laser technology (application of short laser pulses and time-resolved measurement of the receiver signal). These measuring methods and associated mathematical methods for determining optical parameters from such measurement data are state of the art (e.g., Sevick et al., Quantitation of time and frequency resolved optical spectra for the determination of tissue oxygenation, Anal. Biochem. 195, 330-51, 1991; and Patterson et al., Time resolved reflectance and transmittance for the non-invasive measurement of tissue optical properties, Appl. Opt., 28, 2331-36, 1989).

The course of a measurement will be explained here using the example of a provoked allergic reaction (provocation test).

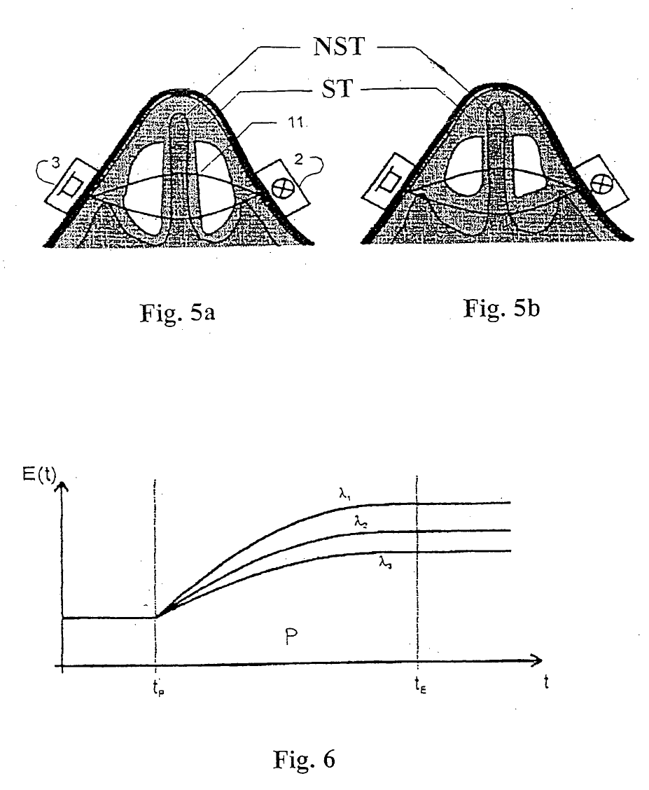

After the person to be examined has been prepared, the application element 1 is fixed on the bridge of the nose near the root of the nose such that the optical emitter element 2 and receiver element 3 are opposite one another on the tissue and so that the optical radiation penetrates as much swellable tissue inside the nose as possible (See FIGS. 3 and 5). Subsequently, an optimal photometric signal is adjusted with the aid of manual, automatic or semi-automatic adjustment of source intensity/intensities and/or detector sensitivity in a range which is suitable for the measurement by way of optomechanical, electronic and/or software methods. The data acquisition is then manually started by the operator. A repeated sequential switching of the radiation sources takes place by the emitter electronics system 15 and simultaneously the measured detector values are acquired by the receiver electronics system 16. This is controlled by the controller 17 inside the basic device 12. Through the separate measurement of the ambient light (dark signal) with light sources switched off, or alternatively through a measurement of the AC portion of a light signal of the light sources that is sufficiently highly modulated, the arrangement ensures that only the light produced by the light sources is measured, and not the ambient light possibly entering the measurement device.

FIG. 6 shows diagrammatically measured extinction values in the course of swelling. The spectral light attenuation values in the unprovoked condition represent the baseline of the measurement. When this is detected in a timeframe of 1 to 2 minutes, an allergenic substance is administered by spraying into one or both nostril(s) and the measurement time is recorded by, e.g., operating a pedal switch at the moment of administration t_{P}. With an allergic reaction, a swelling of the nasal tissue then occurs. This causes a detectable increase in the spectral extinction. FIG. 6 shows spectral extinction values which were standardized at the starting point t_{P} for easier comprehension. At the point of time t_{E} the swelling reaches a stationary condition at which no further swelling is detectable. Only after a time t>t_{E}−t_{P} does the swelling subside again. Diagnostically utilizable information can then be derived from the time response of the spectral extinction values. These include, in particular:
The increase ΔE(λ)=E(λ,t_{E})−E(λ,t_{P}) of the extinction for a wavelength as a gauge of the intensity of the swelling;
The extinction value difference ΔE(λ₁)−ΔE(λ₂) at different wavelengths as a gauge of the increase of the volume proportions of different tissue constituents relative to one another; and
The time difference Δt=t_{E}−t_{P} of the reaction from the moment of provocation P until the stationary final condition as a gauge of the speed of the swelling and the form of the curves E(λ,t) as indicator for the physiological course of the swelling.

### LIST OF REFERENCE NUMBERS

1 Application element
2 Optical emitter element
3 Optical receiver element
4 Current-carrying cable
5 Optical deflection element
6 Optical connection to the emitter element
7 Optical connection to the receiver element
8 Headband
9 Ball joint
10 Attaching clamp
11 Irradiation channel
12 Basic device
13 Light-producing components
14 Light-detecting component
15 Emitter electronic system
16 Receiver electronic system
17 Controller
18 Optical element
NST Non-swellable tissue (bone, cartilage)
ST Swellable Tissue
Provocation

1. An arrangement for optically measuring swelling of a nose, the arrangement comprising:
a device including at least one light-producing component, at least one light-detecting component, an emitter electronics system, a receiver electronics system, and a controller;
at least one optical emitter device connected to the device via at least one first optical connection; and
at least one optical receiver device connected to the device via at least one second optical connection,
wherein the at least one optical emitter device and the at least one optical receiver device are arranged on an application device.

2. The arrangement of claim 1, wherein the at least one first optical connection transmits light produced by the at least one light-producing component.

3. The arrangement of claim 1, wherein the at least one light-producing component produces light which is transmitted by the at least one first optical connection to the at least one optical emitter device.

4. The arrangement of claim 1, wherein the at least one second optical connection transmits light received by the at least one light-detecting component.

5. The arrangement of claim 1, wherein the at least one light-detecting component detects light which is transmitted by the at least one second optical connection to the at least one optical receiver device.

6. The arrangement of claim 1, wherein the at least one light-producing component comprises a plurality of light-producing components.

7. The arrangement of claim 1, wherein the at least one optical emitter device and the at least one optical receiver device are arranged external to the device.

8. The arrangement of claim 1, wherein the application device is structured and arranged to fit onto a nose of a person.

9. The arrangement of claim 8, wherein the at least one optical emitter and receiver devices are structured and arranged to allow the light emitted by the at least one optical emitter device to pass through swellable tissue of at least one side of the nose and to the at least one optical receiver device.

10. The arrangement of claim 1, wherein the application device is capable of being fixed in a form-locking manner to at least an upper part of a nose of a person.

11. The arrangement of claim 1, wherein the at least one light-producing component comprises at least one light source.

12. The arrangement of claim 11, wherein the at least one light source comprises a plurality of light sources emitting light of different wavelengths.

13. The arrangement of claim 1, wherein the at least one light-producing component comprises at least one of an LED, a laser, and a halogen lamp.

14. The arrangement of claim 1, wherein the at least one light-producing component produces a constant optical output.

15. The arrangement of claim 1, wherein the emitter electronics system is structured and arranged to allow the at least one light-producing component to produce a constant optical output.

16. The arrangement of claim 1, wherein the emitter electronics system is structured and arranged to modulate an intensity of the at least one light-producing component.

17. The arrangement of claim 1, wherein the emitter electronics system is structured and arranged to provide for high-frequency modulation of the at least one light-producing component.

18. The arrangement of claim 1, wherein the emitter electronics system provides for light impulses which are less than or equal to a nanosecond.

19. The arrangement of claim 1, wherein the at least one light-detecting component comprises at least one of:
at least one photodetector; and
at least one spectrometer detector.

20. The arrangement of claim 1, wherein the at least one light-detecting component comprises at least one of:
at least one photo semiconductor detector; and
at least one photomultiplier.

21. The arrangement of claim 1, wherein the application device comprises a clamp-shaped body.

22. The arrangement of claim 21, wherein the clamp-shaped body is rigid.

23. The arrangement of claim 21, wherein the clamp-shaped body is slightly flexible.

24. The arrangement of claim 1, wherein the application device comprises two separate elements.

25. The arrangement of claim 1, wherein the application device is capable of being adhesively attached to a nose of a person.

26. The arrangement of claim 1, wherein the application device is capable of being secured to a person via an expansible/length-adjustable strap running around a back of a head of the person.

27. The arrangement of claim 1, wherein the application device is capable of being secured to a spectacle frame.

28. The arrangement of claim 1, further comprising a strap adjustably connected to the application device.

29. The arrangement of claim 1, further comprising at least one optical screening element capable of being arranged around a measuring field.

30. The arrangement of claim 29, wherein the at least one optical screening element comprises one of a light-impervious cap and an optical filter.

31. The arrangement of claim 1, wherein the at least one optical emitter device comprises at least two optical emitter elements and wherein the at least one optical receiver device comprises at least two optical receiver elements.

32. The arrangement of claim 1, wherein the at least one optical emitter device comprises at least two optical emitter elements arranged on one side of the application device and wherein the at least one optical receiver device comprises at least two optical receiver elements arranged on another side of the application device.

33. The arrangement of claim 1, wherein the at least one optical emitter device comprises at least two optical emitter elements adapted to be arranged on one side of a nose of a person and wherein the at least one optical receiver device comprises at least two optical receiver elements adapted to be arranged on another side of the nose of the person.

34. A method of optically measuring swelling of a nose using the arrangement of claim 1, the method comprising:
emitting light with the at least one light-producing component;
transmitting the light, via the at least one first optical connection, from the at least one light-producing component to the at least one optical emitter device;
allowing the light to penetrate through at least one side of the nose;
capturing the light emerging from the nose with the at least one optical receiving device; and
transmitting the light, via the at least one second optical connection, from the at least one optical receiver device to the at least one light-receiving component.

35. The method of claim 34, further comprising recording incoming values that represent a time course of a spectral extinction of optical radiation produced by the at least one light-producing component at selected emission wavelengths before and during a natural swelling of swellable nasal tissue.

36. The method of claim 35, further comprising calculating diagnostically utilizable parameters from the incoming values.

37. The method of claim 34, further comprising recording incoming values that represent a time course of a spectral extinction of optical radiation produced by the at least one light-producing component at selected emission wavelengths before and during a provoked swelling of swellable nasal tissue.

38. The method of claim 37, further comprising calculating diagnostically utilizable parameters from the incoming values.

39. The method of claim 34, wherein the at least one light-producing component comprises different light-producing components producing different emission wavelengths, and wherein the method comprises:
switching, with the emitter electronics system, the different light-producing components, whereby the different light-producing components are switched on brightly one after another.

40. The method of claim 34, further comprising measuring, before optically measuring swelling of the nose, a dark value with non-activated light-producing components to correct for ambient influences.

41. The method of claim 34, further comprising detecting, while optically measuring swelling of the nose, an intensity measurement of the at least one light-producing component.

42. The method of claim 34, further comprising simultaneously detecting, while optically measuring swelling of the nose, an intensity measurement of the at least one light-producing component, whereby the detecting detect an optical output power.

43. The method of claim 34, wherein the light comprises light emitted from a single white-light source, whereby the light is used to differentiate different tissue components

44. The method of claim 34, wherein the at least one light-producing component comprises a single white-light source.

45. The method of claim 34, wherein the at least one optical receiving device comprises a spectrometer detector.

46. The method of claim 34, wherein the at least one light-producing component comprises a plurality of light-producing components, and wherein the method further comprises:
producing, with the light-producing components, light with different carrier frequencies; and
modulating an intensity of the light emitted by the light-producing components.

47. The method of claim 46, further comprising separately detecting, via frequency demultiplexing, light signals.

48. The method of claim 46, wherein the modulating is sinusoidal.

49. The method of claim 34, Wherein the at least one light-producing component comprises a plurality of light-producing components, and wherein the method further comprises:
emitting, with the light-producing components, high-frequency modulated light; and
demodulating light signals in order to determine a phase shift and an amplitude damping.

50. The method of claim 34, wherein the at least one light-producing component comprises a plurality of light-producing components, and wherein the method further comprises:
emitting, with the light-producing components, very short light pulses of less than or equal to one nanosecond; and
determining a temporal photon arrival distribution of received light signals.

51. The method of claim 34, wherein the at least one light-producing component comprises a plurality of light-producing components, and wherein the method further comprises:
emitting, with the light-producing components, very short light pulses of less than or equal to one nanosecond; and
determining, via an optical receiver element, a temporal photon arrival distribution of received light signals,
where by measuring occurs in a time-resolved manner.

52. The method of claim 34, wherein the optically measuring of the swelling of the nose comprises one of:
optically measuring a swelling of each nostril separately; and
optically measuring a swelling of each side of the nose separately.

53. The method of claim 34, wherein the at least one light-producing component comprises a plurality of light-producing components, and wherein the method further comprises:
emitting, with the light-producing components, light of different wavelengths; and
transmitting the light of different wavelengths from the light-producing components to at least one optical emitter device.

54. The method of claim 34, wherein the at least one optical emitter device comprises a plurality of optical emitter devices, and wherein the method further comprises:
arranging the optical emitter devices on each side of the nose; and
transmitting the light from the at least one light-producing component to the optical emitter devices.

55. A system for optically measuring swelling of a nose, the system comprising:
an arrangement including at least one light-producing component, at least one light-detecting component, an emitter electronics device, a receiver electronics device, and a controller;
at least one optical emitter device that can be arranged on a portion of the nose; and
at least one optical receiver device that can be arranged on another portion of the nose,
wherein the at least one optical emitter device is structured and arranged to emit light into the nose, and
wherein the at least one optical receiver device is structured and arranged to capture the light emitted by the at least one optical emitter device.

56. A method of optically measuring swelling of a nose using the system of claim 55, the method comprising:
emitting light with the at least one light-producing component;
transmitting the light from the at least one light-producing component to the at least one optical emitter device;
allowing the light to penetrate through at least one side of the nose;
capturing the light emerging from the nose with the at least one optical receiving device; and
transmitting the light from the at least one optical receiver device to the at least one light-receiving component.

57. A system for optically measuring swelling of a nose, the system comprising:
an arrangement including at least one light-producing component, at least one light-detecting component, an emitter electronics device, a receiver electronics device, and a controller;
at least one optical emitter device that can be arranged on a portion of the nose;
at least one optical receiver device that can be arranged on another portion of the nose; and
a first connection device for transmitting light from the at least one light-producing component to the at least one optical emitter device; and
a second connection device for transmitting light from the at least one optical receiving device to the at least one light-receiving component,
wherein the at least one optical emitter device is structured and arranged to emit light into the nose, and
wherein the at least one optical receiver device is structured and arranged to capture the light emitted by the at least one optical emitter device.

58. A method of optically measuring swelling of a nose using the system of claim 57, the method comprising:
emitting light with the at least one light-producing component;
transmitting the light from the at least one light-producing component to the at least one optical emitter device;
allowing the light to penetrate through at least one side of the nose;
capturing the light emerging from the nose with the at least one optical receiving device; and
transmitting the light from the at least one optical receiver device to the at least one light-receiving component.
